(19) United States
(12) Patent Application Publication
(10) Pub. No.: US 2022/0134026 A1
(43) Pub. Date: May 5, 2022

(54) CONSUMPTION UNIT, INHALER AND MANUFACTURING METHOD

(71) Applicant: HAUNI MASCHINENBAU GMBH, HAMBURG (DE)

(72) Inventors: GUNNAR NIEBUHR, HAMBURG (DE); LENNART KOCK, HAMBURG (DE); THOMAS MÜLLER, HAMBURG (DE); BENJAMIN JUNG, HAMBURG (DE); VOLKMAR VOIGTLÄNDER, HAMBURG (DE); MARKUS SCHMIDT, KAMEN (DE); MATTHIAS GIESE, TOKYO (JP); MICHAEL KLEINE WÄCHTER, LANKAU (DE); MARC KESSLER, HAMBURG (DE)

(21) Appl. No.: 17/431,971

(22) PCT Filed: Feb. 13, 2020
(86) PCT No.: PCT/EP2020/053804

§ 371 Date: Aug. 18, 2021

(30) Foreign Application Priority Data
Feb. 18, 2019 (DE) 10 2019 103 989.4

(51) Int. Cl. A61M 11/04; A24F 40/42; A24F 40/10; A24F 40/485; A24F 7/00; A24F 40/44; A24F 40/70; A24F 40/46

(52) U.S. Cl. CPC A61M 11/042; A24F 40/42; A24F 40/10; A24F 40/485; A61M 2209/045; A24F 40/44; A24F 40/70; A24F 40/46; A24F 7/00

(57) ABSTRACT

A consumption unit for an inhaler comprises a liquid reservoir, a vaporizer device arranged in the liquid reservoir, and a vent having a flow channel extending in the liquid reservoir to a flow connection of the flow channel to the environment. The liquid reservoir comprises a filling opening, which is closed by means of a first closure part.

The present invention relates to a consumption unit for an inhaler comprising a liquid reservoir, a vaporizer device arranged in the liquid reservoir, and a vent having a flow channel extending in the liquid reservoir to a flow connection of the flow channel to the environment. The invention further relates to an inhaler and a method for manufacturing a consumption unit for an inhaler.

Conventional inhalers, such as electronic cigarette products are based on wick-coil technology. Capillary forces transport a liquid from the liquid reservoir along a wick until the liquid is heated by an electrically heated coil and thus vaporized. The wick serves as a liquid-conducting connection between the liquid reservoir and the heating coil comprised by the vaporizer device.

A disadvantage of the wick-coil technology is that an insufficient supply of liquid can lead to local overheating, which can result in the release of pollutants. This is known as “dry puff,” and it must be avoided.

The liquid-conducting connection between the heating coil and the liquid reservoir, as used in wick-coil technology, also has the disadvantage that leakage can easily occur. Thus, even an unfavorable storage of the inhaler and/or pressure fluctuations, for example, as can occur in an airplane, can lead to leakage.

Other generic liquid reservoirs are, for example, closed at one front end and comprise an opening at another front end which is closed by the vaporizer device. The front end closure of the liquid reservoir according to the prior art thus at least partially includes the wick carried through as a liquid conduit from the liquid reservoir to the heating coil and is therefore potentially leaky due to the sealing problem. Furthermore, the liquid reservoir can only be filled if the vaporizer device is not yet mounted in the liquid reservoir or connected thereto in a liquid-conducting manner, as a result of which the assembly steps in the manufacturing process cannot be freely selected in the sense of an economically and manufacturingly optimized sequence.

A generic liquid reservoir is described, for example, in patent application DE 10 2018 206 647.7, which had not yet been disclosed at the time of filing.

The invention is based on the task of providing an improved consumption unit with a more functional structure and/or improved liquid-tightness.

The invention solves the task with the features of the independent claims.

The invention has recognized that it is advantageous that the consumption unit is designed such that the vaporizer device is arranged in the liquid reservoir during filling. It is therefore proposed that the liquid reservoir comprises a filling opening for filling the liquid reservoir with liquid. By enabling filling through the filling opening, the vaporizer device can be mounted in the liquid reservoir during filling. This enables a liquid-tight seal and reliable retainer of the vaporizer device, which is beneficial to the handling and liquid-tightness of the consumption unit, since the wick feedthrough required in the prior art described previously is eliminated.

The filling opening is closed by means of a first closure part to reliably close the filling opening in a liquid-tight manner.The closure of the liquid reservoir by the first closure part may be non-destructively reversibly or irreversibly releasable by the consumer.

Preferably, the filling opening is formed by a free space between the vaporizer device and a wall of the liquid reservoir to provide a practicable embodiment. In this embodiment, when the consumption unit is in a mounted state, there is a free space between the vaporizer device mounted in the liquid reservoir and the wall, which serves as a filling opening and can be closed with the first closure part.

In a preferred embodiment, the liquid reservoir comprises an inner cross-sectional area perpendicular to a longitudinal direction of the flow channel which is larger than an outer cross-sectional area formed by the vaporizer device perpendicular to the longitudinal direction of the flow channel, wherein the filling opening is provided between the inner cross-sectional area of the liquid reservoir and the outer cross-sectional area of the vaporizer device. Due to the different cross-sectional areas, a free space is formed between the vaporizer device and the wall of the liquid reservoir, which comprises the size and shape of the difference between the inner cross-sectional area of the liquid reservoir respectively the inner contour and the outer cross-sectional area of the vaporizer device respectively the outer contour. The inner cross-sectional area of the liquid reservoir is thereby the cross-sectional area of the liquid reservoir at the location where the vaporizer device is arranged and/or retained. The outer cross-sectional area of the vaporizer device is given by the circumference or contour of the vaporizer device in the plane of the inner cross-sectional area of the liquid reservoir. In the present embodiment, it is sufficient that the vaporizer device comprises, at at least one location of its circumference, a diameter equal to the diameter of the cross-section of the liquid reservoir to be retained within the liquid reservoir. At at least one other location of the circumference, the vaporizer device comprises a diameter which is smaller than the diameter of the free inner cross-sectional area of the liquid reservoir in order to form the free space or the filling opening.

Advantageously, the inner cross-sectional area of the liquid reservoir is round and the outer cross-sectional area of the vaporizer device is oval to provide an effective embodiment. For example, the outer cross-section of the vaporizer device may be elliptical and a major semi-axis of the outer cross-section of the vaporizer device may be equal or nearly equal to the diameter of the inner cross-sectional area of the liquid reservoir. In this case, the vaporizer device would be supported at two points opposite to each other on the major axis of the oval-shaped outer cross-sectional area of the vaporizer device. At the same time, there would be one opening at each of two points on the minor axis opposite the oval-shaped outer cross-sectional area of the vaporizer device. One opening may be the filling opening, while the other opening may serve to vent the liquid reservoir during filling. Other oval or non-elliptical outer cross-sections of the vaporizer device are also conceivable. In this case, the openings can preferably be arranged and shaped centrically and/or symmetrically to one another. However, arrangements and shapes deviating from this are not excluded by the invention. In particular, the openings can be individually shaped and arranged for an optimal filling process and/or for a geometrically favorable shaping of the closure part. In particular, the openings can be arranged off-center if, for example, filling is to be performed laterally of the vaporizer device.

Preferably, the filling opening is formed by a bore in a wall of the liquid reservoir in order to provide a punctual and/or easily closable filling opening.

In an advantageous embodiment, a venting opening is provided, which is closed by means of a second closure part, in order to simplify the filling of the consumption unit by the possibility of venting and, at the same time, to promote the liquid-tightness of the consumption unit through closing by the second closure part.

Preferably, the filling opening is provided between the vent and a wall of the liquid reservoir, thus eliminating a special manufacturing operation of the opening and allowing a practicable embodiment to be realized.

It is preferred that the vent and the vaporizer device and/or the vent and the liquid reservoir are formed at least in sections as a one-piece component, in order to reduce the number of components by the one-piece design and at the same time to avoid a potential leakage at a joint between the vaporizer device and/or the vent and the liquid reservoir, which is avoided by the one-piece design. The vent and the vaporizer device may be one-piece and adapted to be arranged in the liquid reservoir. The vent and the liquid reservoir may also be one-piece, wherein the vaporizer device is adapted to be arranged in the one-piece component comprising the vent and the liquid reservoir.

Particularly advantageously, the liquid reservoir and/or the vaporizer device is molded with a portion that at least partially forms the vent to provide a cost-effective and reliable consumption unit with an at least in sections one-piece component.

Preferably, the first closure part fixes the vaporizer device relative to the liquid reservoir in order to provide a properly assembled and liquid-tight consumption unit by the fixation. The closure part can secure the arrangement of the vaporizer device in the liquid reservoir against displacement and/or rotation.

Preferably, the first closure part comprises a coupling geometry adapted to the geometry of the liquid reservoir and/or the vaporizer device in order to advantageously mechanically retain the first closure part and to prevent leakage between the first closure part and the liquid reservoir and/or the vaporizer device.

Advantageously, the vent is fluidically connected to an outlet side of the vaporizer device to be able to form an advantageous flow channel through which air, vapor and/or aerosol can flow.

Preferably, the first closure part is designed as a mouthpiece in order to provide an embodiment with only a few components. The first closure part thus serves as a mouthpiece on which the consumer draws to inhale, i.e. applies a negative pressure to the inhaler. This allows easy assembly of the consumption unit or inhaler and/or can improve the possibility of cleaning the mouthpiece. Due to the design as a mouthpiece, the first closure part can be particularly easy to handle. When inserting the consumption unit, the mouthpiece can first push out and/or pierce a previously existing, temporary closure part and close the resulting free space itself, as it is known, for example, from ink cartridges. The consumption unit is thus first pierced by the mouthpiece and then closed again by the mouthpiece itself.

In an advantageous embodiment, the first closure part comprises a flow channel which connects the flow channel of the vent with the environment in order to provide a fluidic connection between the vent and the environment. Thus, the first closure part can perform the function of the mouth end of the inhaler. Furthermore, the flow channel of the vent is thereby extended and the gas or aerosol and/or vapor to be inhaled can be further cooled down to a lower temperature.

It is further proposed that a wick structure is provided between the liquid reservoir and the vaporizer device, and the wick structure is oriented and/or connected to the liquid reservoir such that the feed of liquid through the wick structure is perpendicular or at an angle not equal to 180 degrees to the longitudinal extension of the vaporizer device. By the proposed solution, the liquid is deflected in the transition to the vaporizer device and transported away at an angle. In this way, a swirling of the liquid can also be created, which supports or promotes the vaporization process. Furthermore, the wick structure can thus also be arranged at a structurally more favorable location, insofar as a parallel feed is not possible, for example, due to a limited length of the consumption unit.

The invention also comprises a method for manufacturing a consumption unit for an inhaler comprising a liquid reservoir, a vaporizer device and a first closure part.

It is proposed that the following process steps are performed in the following order: Inserting the vaporizer device into the liquid reservoir to a final fastening position, filling the liquid reservoir with liquid through a filling opening, and closing the filling opening by means of the first closure part. This method makes use of the advantages described above, which result from the invention. In particular, the consumption unit can be reliably closed in a liquid-tight manner after filling.

Advantageously, the vaporizer device comprises a smaller outer cross-sectional area or also outer contour perpendicular to the insertion direction than the free inner cross-sectional area of the liquid reservoir or also inner contour perpendicular to the insertion direction of the vaporizer device, and the filling opening is formed by the free space, which is formed due to the smaller outer contour of the vaporizer device between the vaporizer device and the liquid reservoir, in order to specify a method in which a free space for filling is formed in a defined manner during assembly as a filling opening which can be closed by the first closure part without requiring a separate manufacturing process for this purpose.

The invention is explained below on the basis of preferred embodiments with reference to the accompanying figures. Thereby shows

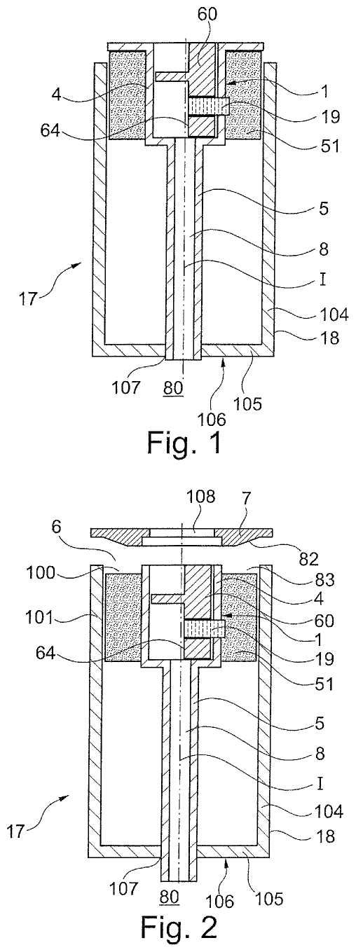

FIG. 1 a section through a consumption unit described in application DE 10 2018 206 647.7;

FIGS. 2-4 each a section through a consumption unit described in application DE 10 2018 206 647.7;

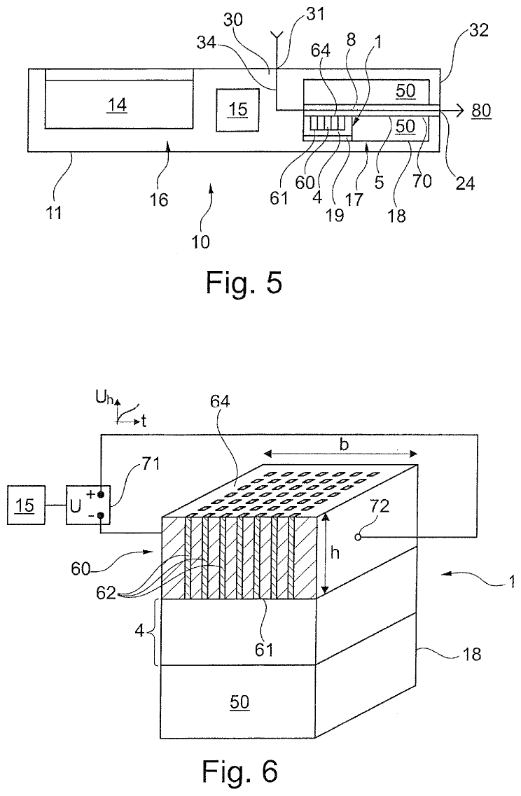

FIG. 5 a schematic illustration of an inhaler;

FIG. 6 a perspective cross-sectional view of a heater body with a liquid reservoir; and

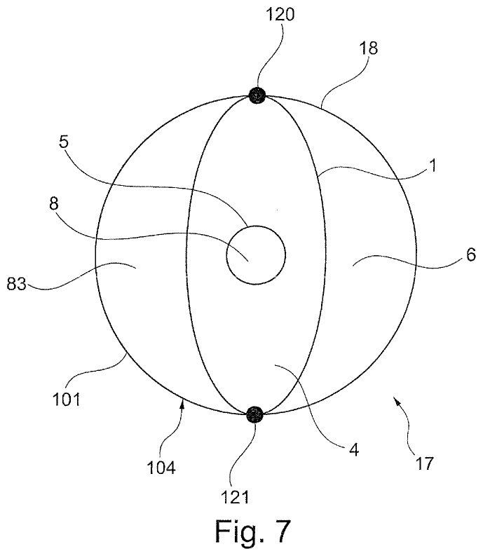

FIG. 7 a schematic cross-section of an embodiment of a consumption unit according to the invention.

FIGS. 1 to 4 each show a longitudinal section through a consumption unit 17, their common features are outlined below.

The consumption unit 17 comprises a liquid reservoir 18 for storing liquid 50 to be vaporized, as shown in FIGS. 5 and 6. In the embodiments shown in FIGS. 1 to 4, the liquid reservoir 18 is cylindrical in shape. The liquid reservoir 18 comprises a base surface 105 at one front end 106, which can have any contour. From the base surface 105, preferably a jacket surface 104 extends circumferentially along preferably a direction or axis perpendicular to the base surface 105, which defines a longitudinal direction I. The base surface 105 and the jacket surface 104 define the volume of the cylindrical liquid reservoir 18.

In the exemplary case of a circular cylindrical fluid reservoir 18, the base surface 105 comprises a round contour. However, it is also conceivable that the liquid reservoir 18 comprises a non-round cross-section respectively the base surface 105 is non-round. For example, the liquid reservoir 18 and/or the base surface 105 may comprise one or more corners.

The consumption unit 17 comprises a vaporizer device 1 arranged in the liquid reservoir 18 for vaporizing liquid 50 stored in the liquid reservoir 18. The vaporizer device 1 is explained in more detail with reference to FIGS. 5 and 6.

The consumption unit 17 comprises a vent 5 with a flow channel 8 provided in the interior of the vent 5 for transporting air, aerosol and/or vapor, see FIGS. 1 to 4. The vent 5 extends through the liquid reservoir 18 from the vaporizer device 1 to a flow connection of the flow channel 8 to the environment 80. The vent 5 comprises a smaller cross-section than the jacket surface 104 of the liquid reservoir 18. Thus, the vent 5 may be arranged within the fluid reservoir 18. Advantageously, the vent 5 and/or the flow channel 8 extends parallel to the jacket surface 104 of the liquid reservoir 18. It is particularly advantageous that the vent 5 and/or the flow channel 8 extends centrally through the liquid reservoir 18. Preferably, the vent 5 is shaped like a hollow cylinder and/or extends along the longitudinal axis I.

In the embodiments shown, the vent 5 and the vaporizer device 1 are formed as a one-piece component. For example, the vent 5 may be molded to a carrier 4 of the vaporizer device 1. This makes leakage between the vaporizer device 1 and the vent 5 impossible and simplifies handling.

In other embodiments not shown, the vent 5 and the liquid reservoir 18 may be formed as a one-piece component, for example by the vent 5 being molded to the liquid reservoir 18. In these embodiments, the vent 5 is molded onto an end face of the liquid reservoir 18, for example, onto the base surface 105. Thus, leakage between the liquid reservoir 18 and the vent 5 is impossible.

The vent 5 is fluidically connected to an outlet side 64 of the vaporizer device 1 to allow aerosol and/or vapor flowing through the vent 5 to be directed along the flow channel 8 into the environment 80. In operation, the environment 80 is formed by the mouth of the consumer drawing on a mouth end 32 of the inhaler 10 for inhalation. Such an inhaler 10 may be used, for example, to administer medical and/or health-promoting substances. Furthermore, the inhaler 10 may also serve to provide enjoyment of flavored aerosols, for example, as is the case with electronic cigarettes.

FIG. 1 shows a consumption unit 17 according to the application DE 10 2018 206 647.7 not yet published at the time of filing of this invention. The vent 5 extends through a piercing opening 107 provided in the base surface 105. The piercing opening 107 must be very precisely dimensioned in order to be able to realize the liquid-tightness between the liquid reservoir 18 and the vent 5 and/or the vent 5 in the liquid reservoir 18. Additionally, a molded seal may be provided which is inherently flexible or elastic so that the manufacturing accuracy requirements can be reduced. The liquid-tightness is then realized by the sealing contact of the seal, wherein in addition shape deviations or minor unevenness can be compensated.

For filling the liquid reservoir 18, the vent 5 must be inserted into the liquid reservoir 18 to such an extent that the piercing opening 107 is closed in a liquid-tight manner, but only to such an extent that an opening for filling the liquid reservoir 18 remains on the side opposite the end face 106 with the piercing opening 107 (not shown).

Subsequently, the vent 5 and the vaporizer device 1 must be inserted into the liquid reservoir 18 to such an extent so that the vaporizer device 1 can close the liquid reservoir 18 in a liquid-tight manner.

The embodiment in FIG. 1 does not comprise a first closure part 7. According to this solution, a first closure part 7 is not needed, since the liquid reservoir 18 is closed by the vaporizer device 1 itself.

In the embodiment according to the invention shown in FIG. 2, the consumption unit 17 comprises a piercing opening 107 in the base surface 105. The vent 5 extends through the liquid reservoir 18 from the vaporizer device 1 to the piercing opening 107. The vent 5 is mechanically fixed in the piercing opening 107 and closes the piercing opening 107 in a liquid-tight manner.

The liquid reservoir 18 comprises a filling opening 6 for filling the liquid reservoir 18 with a liquid 50. The filling opening 6 is formed by a free space 100 between the vaporizer device 1 or the carrier 4 of the vaporizer device 1 and a wall 101, as can be seen in FIG. 2. In this embodiment, the wall 101 is the jacket surface 104 of the liquid reservoir 18.

The filling opening 6 is closable with a first closure part 7 in order to be able to close the liquid reservoir 18 in a liquid-tight manner, see FIG. 2. The first closure part 7 mechanically fixes the vaporizer device 1 relative to the liquid reservoir 18 in order to provide a reliable retainer of the vaporizer device 1.

The first closure part 7 comprises a coupling geometry 82 adapted to the geometry of the liquid reservoir 18 and/or the vaporizer device 1, which allows easy mounting of the first closure part 7. In this example, the first closure part 7 is adapted to the geometry such that the first closure part 7 comprises an interface 108 that can serve to supply air into the vaporizer device 1. Furthermore, the interface 108 can serve to support the carrier 4 of the vaporizer device 1 resp. the vaporizer device 1. Furthermore, the first closure part 7 may comprise retaining elements provided for connection, such as catches, clips, recesses, protrusions and/or the like, wherein the liquid reservoir 18 and/or the vaporizer device 1 may comprise retaining elements belonging to the retaining elements.

Herein, the interface 108 is formed by a recess that allows for an air supply. The recess may comprise a shape corresponding to the outer shape of the vaporizer device 1, so that it closes the outer shape of the vaporizer device 1 in a liquid-tight manner and fixes the vaporizer device 1.

In the embodiment according to the invention shown in FIG. 3, the consumption unit 17 comprises a piercing opening 107 in the base surface 105. The vaporizer device 1 is mechanically fixed in the piercing opening 107 and closes the piercing opening 107 in a liquid-tight manner. The vent 5 extends from the vaporizer device 1 through the liquid reservoir 18 to a side of the liquid reservoir 18 opposite the base surface 105.

The filling opening 6 is provided between the vent 5 and a wall 101 of the liquid reservoir 18. In this embodiment, the wall 101 is formed by the jacket surface 104.

The first closure part 7 holds the vaporizer device 1 in place relative to the liquid reservoir 18 against tilting, as the first closure part 7 holds the vent 5 in place. Furthermore, the first closure part 7 can fasten the vaporizer device 1 with respect to a displacement along the longitudinal axis I. For this purpose, for example, a stop, an interference fit or also an adhesive connection can be provided individually or in combination.

The first closure part 7 comprises a coupling geometry 82 adapted to the geometry of the fluid reservoir 18, as explained with reference to FIG. 2.

In the embodiment shown in FIG. 3, the first closure part 7 is formed as a mouthpiece 81. The consumer can thus directly engage the first closure part 7 and create a negative pressure by inhaling, which leads to an air flow through the flow channel 8. For this purpose, the mouthpiece 81 may comprise a shape corresponding to a mouth end 32 of the inhaler 10 and/or form the mouth end 32 of the inhaler 10. In this regard, the consumption unit 17 may comprise a temporary closure part (not shown) which is discharged and/or pierced by the first closure part 7 formed as the mouthpiece 81. The mouthpiece 81 subsequently closes the opening created in this way and then forms the first closure part 7.

The first closure part 7 comprises a flow channel 103, which connects the flow channel 8 of the vent 5 to the environment 80. The flow channel 103 may be formed by an opening in the first closure part 7, which also serves to fix the vent 5, and/or through which the vent 5 preferably projects substantially flush.

In the embodiments of FIGS. 2 and 3, the first closure part 7 is a component separate from the vaporizer device 1. Thus, the consumer may remove the first closure part 7 for filling the liquid reservoir 18 to thereby expose the filling opening 6 and, after filling the liquid reservoir 18 with liquid 50, close the filling opening 6 again with the first closure part 7.

In addition to the filling opening 6, a venting opening 83 can be provided, as explained in FIGS. 2, 3 and 7, which can be additionally closed by means of the first closure part 7.

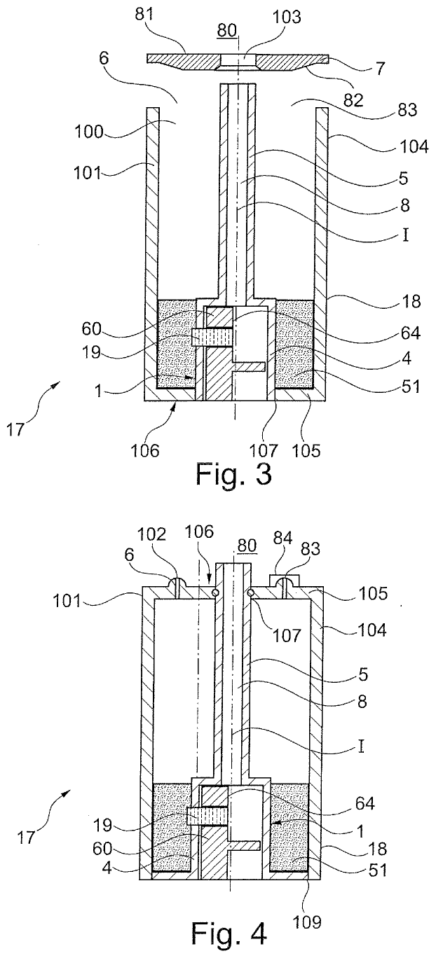

In the embodiment according to the invention shown in FIG. 4, the consumption unit 17 comprises a passage opening 109 in a side of the liquid reservoir 18 opposite to the base surface 105. The vaporizer device 1 is mechanically fixed in the passage opening 109 anti closes the passage opening 109 in a liquid-tight manner.

The consumption unit 17 comprises a piercing opening 107 in the base surface 105. The vent 5 is mechanically fixed in the piercing opening 107 and closes the piercing opening 107 in a liquid-tight manner.

The vent 5 extends through the liquid reservoir 18 from the vaporizer device 1 to the base surface 105 of the liquid reservoir 18.

The filling opening 6 is formed by a bore 102 in a wall 101 of the liquid reservoir 18. The bore 102 is arranged on the base surface 105. The bore 102 may, for example, be the result of a piercing of a cannula used to fill the fluid reservoir 18 or may be made in some other way, for example by drilling into the base surface 105. However, the bore 102 may also be provided in another wall 101 of the liquid reservoir 18, provided that this is more convenient for the filling or closing process.

The first closure part 7 is not explicitly shown in FIG. 4. The first closure part 7 may be a cap-like component and/or a component separate from the liquid reservoir 18, as shown in FIGS. 2 and 3, which closes the filling opening 6. However, the first closure part 7 may also be formed by the base surface 105 of the liquid reservoir 18 itself, in which the liquid reservoir 18 is formed, at least in the region of the filling opening 6, in such a way that the filling opening 6 advantageously closes automatically after filling without any further component. For example, the filling opening 6 can close after filling by selecting a correspondingly elastic material and/or with the aid of a heat treatment. It would be conceivable, for example, to use an openable and automatically reclosable membrane as the first closure part 7.

A venting opening 83 is provided, which is closed by means of a second closure part 84. The second closure part 84 is shown only schematically in FIG. 4. In other embodiments, the second closure part 84 can be designed in the same way as the first closure part 7.

In an alternative embodiment to FIG. 4, the filling opening 6 and the venting opening 83 can be closed by a common first closure part 7.

In an alternative embodiment to FIG. 4, the filling opening 6 and/or the venting opening 83 may be provided on the end face of the fluid reservoir 18 opposite the base surface 105. In particular, the filling opening 6 and/or the venting opening 83 may be provided in the carrier 4 of the vaporizer device 1. Furthermore, the filling opening 6 and/or the venting opening can also be provided in the jacket surface 104 resp. wall 101 of the liquid reservoir 18.

FIG. 5 schematically shows an inhaler 10. The inhaler 10, in this case an inhaler in the form of an electronic cigarette product, comprises a housing 11 in which an air channel 30 is provided between at least one air inlet opening 31 and an air outlet opening 24 at a mouth end 32 of the inhaler 10. The mouth end 32 of the inhaler 10 thereby refers to the end at which the consumer draws for the purpose of inhalation, thereby applying a negative pressure to the inhaler 10 and generating an air flow 34 in the air channel 30.

The inhaler 10 advantageously consists of a base part 16 and a consumption unit 17, which comprises the vaporizer device 1 and the liquid reservoir 18 and is designed in particular in the form of a replaceable cartridge. The air sucked in through the inlet opening 31 is conducted in the air channel 30 to, or through, the at least one vaporizer device 1. The vaporizer device 1 is connected or connectable to the liquid reservoir 18, in which at least one liquid 50 is stored.

The vaporizer device 1 vaporizes liquid 50, which is advantageously fed to the vaporizer device 1 from the liquid reservoir 18 by a wick or wick structure 19 by means of capillary forces, and discharges the vaporized liquid as an aerosol/vapor into the air stream 34 at an outlet side 64.

On an inlet side 61 of the heating body 60, the porous and/or capillary, liquid-conducting wick structure 19 is advantageously arranged, as shown schematically in FIG. 5. The connection of the wick structure 19 to the liquid reservoir 18 and to the heating body 60 via the carrier 4 as shown in FIG. 5 is to be understood only as an example. In particular, a fluid interface and/or multiple fluid lines may be provided between fluid reservoir 18 and wick structure 19. Thus, the fluid reservoir 18 may be spaced apart from the wink structure 19. The wink structure 19 contacts the inlet side 61 of the heating body 60 advantageously in a planar manner and covers all passage openings 62 on the inlet side. On the side opposite the heating body 60, the wick structure 19 is connected to the liquid reservoir 18 in a liquid-conducting manner. The liquid reservoir 18 may be larger in dimensions than the wick structure 19. For example, the wick structure 19 may be inserted into an opening of a housing of the liquid reservoir 18. A plurality of vaporizer devices 1 may also be associated with a liquid reservoir 18. The wick structure 19 may be generally one-piece or multi-piece.

The wick structure 19 comprises porous and/or capillary material which, due to capillary forces, is capable of passively re-feeding liquid vaporized by the heating body 60 from the liquid reservoir 18 to the heating body 60 in sufficient quantity to prevent the passage openings 62 from running dry and resulting problems.

Advantageously, the wick structure 19 comprises an electrically non-conductive material to prevent undesirable heating of liquid in the wick structure 19 by current flow. The wick structure 19 advantageously comprises a low thermal conductivity.

The wick structure 19 advantageously comprises one or more of the materials: cotton, cellulose, acetate, glass fiber fabric, glass fiber ceramic, sintered ceramic, ceramic paper, aluminosilicate paper, metal foam, metal sponge, another heat-resistant, porous and/or capillary material having a suitable feed rate, or a composite of two or more of the foregoing materials. In an advantageous practical embodiment, the wick structure 19 may comprise at least a ceramic fiber paper and/or a porous ceramic. The volume of the wick structure 19 is preferably in the range between 1 mm³ and 10 mm³, further preferably in the range between 2 mm³ and 8 mm³, still further preferably in the range between 3 mm³ and 7 mm³ and is for example 5 mm³.

If the wick structure 19 is made of an electrically and/or thermally conductive material, which is not excluded, an insulating layer of an electrically and/or thermally insulating material, for example glass, ceramic or plastic, is advantageously provided between the wick structure 19 and the heating body 60, with openings extending through the insulating layer and corresponding to the passage openings 62. The wick structure 19 may preferably be oriented and/or connected to the liquid reservoir 18 such that the feed of liquid through the wick structure 19 is perpendicular or at an angle not equal to 180 degrees to the longitudinal extent of the vaporization device 1. The liquid is thereby deflected starting from the wick structure 19 in the transition to the vaporizer device and transported away from the vaporizer device 1 at an angle of, for example, 90 degrees or at an angle between 0 and 90 degrees, wherein the vaporization of the liquid and the drawing force of the consumer during inhalation causes and supports the transport away of the liquid.

An advantageous volume of the liquid reservoir 18 is in the range between 0.1 ml and 5 ml, preferably between 0.5 ml and 3 ml, further preferably between 0.7 ml and 2 ml or 1.5 ml.

The inhaler 10, which in the present embodiment is formed by an electronic cigarette, further comprises an electrical energy storage unit 14 and an electronic control device 15. The energy storage unit 14 is generally arranged in the base part 16 and may in particular be a disposable electro-chemical battery or a rechargeable electro-chemical battery, for example a lithium-ion battery. The consumption unit 17 is arranged between the energy storage unit 14 and the mouth end 32. The electronic control device 15 comprises at least one digital data processing device, in particular microprocessor and/or microcontroller, in the base part 16 (as shown in FIG. 5) and/or in the consumption unit 17.

A sensor, for example a pressure sensor or a pressure or flow switch, is advantageously arranged in the housing 11, wherein the control device 15 can determine, based on a sensor signal output by the sensor, that a consumer is drawing on the mouth end 32 of the inhaler 10 to inhale. In this case, the control device 15 controls the vaporizer device 1 to feed liquid 50 from the liquid reservoir 18 as an aerosol/vapor into the air stream 34.

The vaporizer device 1 or the at least one vaporizer 60 is arranged in a part of the consumption unit 17 facing away from the mouth end 32. This enables effective electrical coupling and control of the vaporizer device 1. Advantageously, the air flow 34 passes through an air channel 70 extending axially through the liquid reservoir 18 to the air outlet opening 24.

The liquid 50 stored in the liquid reservoir 18 to be dispensed is, for example, a mixture of 1,2-propylene glycol, glycerol, water, at least one aroma (flavour) and/or at least one active ingredient, in particular nicotine. However, the indicated components of the liquid 50 are not mandatory. In particular, flavoring and/or active ingredients, in particular nicotine, can be left out.

The consumption unit resp. cartridge 17 or the base part 16 advantageously comprise a non-volatile data memory for storing information or parameters relating to the consumption unit or cartridge 17. The data memory can be part of the electronic control device 15. The data memory advantageously stores information on the composition of the liquid stored in the liquid reservoir 18, information on the process profile, in particular power/temperature control; data on condition monitoring or system testing, for example leak testing; data relating to copy protection and counterfeit protection, an ID for unambiguous identification of the consumption unit resp. cartridge 17, serial number, date of manufacture and/or expiration date, and/or number of draws (number of inhalation draws by the consumer) or the time of use. The data memory is advantageously electrically connected or connectable to the control device 15.

In the inhaler 10 and/or in an external memory, which can be connected to the inhaler 10 in a suitable and per se known manner, at least temporarily, by means of communication technology, user-related data, in particular about the smoking behavior, could also be stored and preferably also used for controlling and regulating the inhaler 10.

FIG. 6 shows a vaporizer device 1. The vaporizer device 1 comprises a block-shaped, preferably monolithic heating body 60 preferably made of an electrically conductive material, in particular a semiconductor material preferably silicon, and a carrier 4. It is not necessary that the entire heating body 60 consists of an electrically conductive material. It may be sufficient, for example, that the surface of the heating body 60 is electrically conductive, for example metallic, coated or preferably suitably doped. In this case, the entire surface need not be coated; for example, metallic or preferably non-metallic or non-metallically laminated metallic conductor tracks may be provided on a non-conductive or semi-conductive base body. It is also not essential that the entire heating body 60 heats; for example, it may be sufficient if a portion or a heating layer of the heating body 60 heats in the region of the outlet side 64.

The heating body 60 is provided with a plurality of microchannels or passage openings 62, which connect an inlet side 61 of the heating body 60 to an outlet side 64 of the heating body 60 in a liquid-conducting manner. The inlet side 61 is connected in a liquid-conducting manner to the liquid reservoir 18 via a wick structure 19 not shown in FIG. 6. The wick structure 19 serves to passively feed liquid from the liquid reservoir 18 to the heating body 60 by means of capillary forces.

The average diameter of the passage openings 62 is preferably in the range between 5 μm and 200 μm, further preferably in the range between 30 μm and 150 μm, still further preferably in the range between 50 μm and 100 μm. Due to these dimensions, a capillary effect is advantageously generated, so that liquid entering a passage opening 62 at the inlet side 61 rises upward through the passage opening 62 until the passage opening 62 is filled with liquid. The volume ratio of passage openings 62 to heating body 60, which may be referred to as the porosity of the heating body 60, is for example in the range between 10% and 50%, advantageously in the range between 15% and 40%, still further advantageously in the range between 20% and 30%, and is for example 25%.

The edge lengths of the areas of the heating body 60 provided with passage openings 62 are, for example, in the range between 0.5 mm and 3 mm, preferably between 0.5 mm and 1 mm. The dimensions of the areas of the heating body 60 provided with passage openings 62 can be, for example: 0.95 mm×1.75 mm or 1.9 mm×1.75 mm or 1.9 mm×0.75 mm. The edge lengths of the heating body 60 may be, for example, in the range between 0.5 mm and 5 mm, preferably in the range between 0.75 mm and 4 mm, further preferably in the range between 1 mm and 3 mm. The area of the heating body 60 (chip size) can be, for example, 1 mm×3 mm, 2 mm×2 mm or 2 mm×3 mm.

The width b of the heating body 60 (see FIG. 6) is preferably in the range between 1 mm and 5 mm, further preferably in the range between 2 mm and 4 mm, and is for example 3 mm. The height h of the heating body 60 (see FIG. 6) is preferably in the range between 0.05 mm and 1 mm, further preferably in the range between 0.1 mm and 0.75 mm, still further preferably in the range between 0.2 mm and 0.5 mm, and is for example 0.3 mm. Even smaller heating bodies 60 can also be manufactured, provided and functionally operated.

The number of passage openings 62 is preferably in the range between four and 1000. In this way, the heat input into the passage openings 62 can be optimized and a reliable high vaporization capacity as well as a sufficiently large steam outlet area can be realized.

The passage openings 62 are arranged in the form of a square, rectangular, polygonal, circular, oval or other shaped array. The array may be in the form of a matrix with s columns and z rows, wherein s is advantageously in the range between 2 and 50 and further advantageously in the range between 3 and 30 and/or z is advantageously in the range between 2 and 50 and further advantageously in the range between 3 and 30. In this way, an effective and easily producible arrangement of the passage openings 62 with reliable high vaporization performance can be realized.

The cross-section of the passage openings 62 may be square, rectangular, polygonal, round, oval or otherwise shaped, and/or may change section-wise in the longitudinal direction, in particular increase, decrease or remain constant.

The length of one or each passage opening 62 is preferably in the range between 100 μm and 1000 μm, further preferably in the range between 150 μm and 750 μm, still further preferably in the range between 180 μm and 500 μm and is for example 300 μm. In this way, optimum liquid absorption and portion formation can be achieved with sufficiently good heat input from the heating body 60 into the passage openings 62.

The distance between two passage openings 62 is preferably at least 1.3 times the clear diameter of a passage opening 62, wherein the distance is related to the center axes of the two passage openings 62. The distance can preferably be 1.5 to 5 times, more preferably 2 to 4 times, the clear diameter of a passage opening 62. In this way, an optimal heat input into the heating body 60 and a sufficiently stable arrangement and wall thickness of the passage openings 62 can be realized.

Based on the features described above, the heating body 60 may also be referred to as a volume heater.

The vaporizer device 1 comprises a heating voltage source 71 which is preferably controllable by the control device 29 and is connected to the heating body 60 via electrodes 72 on opposite sides of the heating body 60, so that an electrical voltage Uh generated by the heating voltage source 71 results in a current flow through the heating body 60. Due to the ohmic resistance of the electrically conductive heating body 60, the current flow causes heating of the heating body 60 and therefore vaporization of liquid contained in the passage openings 62. Vapor/aerosol 6 generated in this manner escapes to the outlet side 64 from the passage openings 62 and is mixed into the air flow 34, see FIG. 5. More precisely, on detecting an air flow 34 caused by drawing of the consumer through the air channel 30, the control device 29 controls the heating voltage source 71, wherein the liquid contained in the passage openings 62 is driven out of the passage openings 62 in the form of vapor/aerosol 6 by spontaneous heating.

In this case, the duration of the individual vaporization steps at different temperatures and/or a vaporization of the individual components of the individual portions of the liquid can be kept short and/or can be timed with a drive frequency such that the step-by-step vaporization cannot be perceived by a consumer and a largely homogeneous, taste-conform, repeatably precise aerosol formation can nevertheless be ensured. In particular, a lighter-boiling component of the liquid is advantageously vaporized first in a first vaporization interval at a first temperature A and then a higher-boiling component of the liquid is vaporized in a second vaporization interval at a second temperature B, which exceeds the temperature A.

An electronic or electrical connection of the heating body 60 can be made, for example, via clamping, spring or press contacts, wirebonding and/or soldering.

Preferably, a voltage curve Uh(t) adapted to the liquid mixture used is stored in the data memory of the inhaler 10. This makes it possible to preset the voltage curve Uh(t) adapted to the liquid used, so that the heating temperature of the heating body 60, and thus also the temperature of the capillary passage openings 62, can be controlled over the vaporization process in accordance with the known vaporization kinetics of the respective liquid, whereby optimum vaporization results can be achieved. The vaporization temperature is preferably in the range between 100° C. and 400° C., further preferably between 150° C. and 350° C., still further preferably between 190° C. and 290° C.

The heating body 60 can advantageously be made from portions of a wafer with thin film layer technology, which comprises a layer thickness preferably less than or equal to 1000 μm, further preferably 750 μm, still further preferably less than or equal to 500 μm. Surfaces of the heating body 60 may advantageously be hydrophilic. The outlet side 64 of the heating body 60 may advantageously be microstructured or comprise micro grooves.

The vaporizer device 1 is adjusted to dispense a quantity of liquid preferably in the range between 1 μl and 20 μl, further preferably between 2 μl and 10 μl, still further preferably between 3 μl and 5 μl, typically 4 μl per puff of the consumer. Preferably, the vaporizer device 1 can be adjustable with respect to the amount of liquid/vapor per puff, i.e. per puff duration from 1 s to 3 s.

In the following, the sequence of the vaporization process is explained by way of example.

In an initial state, the voltage source 71 or the energy storage 14 is switched off for the heating process.

To vaporize liquid 50, voltage source 14, 71 for heating body 60 is activated. The voltage Uh is adjusted so that the evaporation temperature in the heating body 60 and thus in the passage openings 62 is adapted to the individual vaporization behavior of the liquid mixture used. This prevents the risk of local overheating and thereby the formation of pollutants.

In particular, undesirable differential vaporization of a liquid mixture can also be counteracted or avoided. A liquid mixture could otherwise lose components prematurely due to different boiling temperatures in the course of a sequence of vaporization processes, in particular “puffs”, before the reservoir 18 of the liquid 50 is completely emptied, which could result in undesirable effects during operation, such as a lack of consistency of dosage for a user, in particular for a pharmaceutically active liquid.

Once an amount of liquid equal to or related to the volume of the passage openings 62 is vaporized, the heating voltage source 71 is deactivated. Since the liquid properties and quantity are advantageously known exactly and the heating body 60 comprises a measurable temperature-dependent resistance, this point in time can be determined or controlled very precisely. The energy consumption of the vaporizer device 1 can therefore be reduced compared to known devices, since the required vaporization energy can be introduced in a more metered and thus more precise manner.

After completion of the heating process, the passage openings 62 are predominantly or completely emptied. The heating voltage 71 is then kept switched off until the passage openings 62 are filled again by means of liquid refeed through the wick structure 19. As soon as this is the case, the next heating cycle can be started by switching on the heating voltage 71.

The driving frequency of the heating body 60 generated by the heating voltage source 71 is generally advantageously in the range of 1 Hz to 50 kHz, preferably in the range of 30 Hz to 30 kHz, still more advantageously in the range of 100 Hz to 25 kHz.

The frequency and duty factor of the heating voltage Uh for the heating body 60 are advantageously adapted to the natural oscillation or natural frequency of the bubble oscillations during bubble boiling. Advantageously, the period 1/f of the heating voltage can therefore be in the range between 5 ms and 50 ms, further advantageously between 10 ms and 40 ms, still further advantageously between 15 ms and 30 ms, and for example 20 ms. Depending on the composition of the vaporized liquid 50, frequencies other than those mentioned can be optimally adapted to the natural oscillation or natural frequency of the bubble oscillations.

Furthermore, it has been found that the maximum heating current generated by the heating voltage Uh should preferably be no more than 7 A, further preferably no more than 6.5 A, still further preferably no more than 6 A, and optimally in the range between 4 A and 6 A, in order to ensure concentrated vapor while avoiding overheating.

The feed rate of the wick structure 19 is again optimally adapted to that of the vaporization rate of the heating body 60, so that sufficient liquid 50 can be refed at any given time and running empty of the region in front of the heating body 60 is avoided.

The vaporizer device 1 is preferably based on MEMS technology, in particular made of silicon, and is therefore advantageously a micro-electro-mechanical system.

According to the above, it is advantageously proposed a layered structure consisting of a heating body 60 based on Si, which is advantageously planar at least on the inlet side 61, and one or more underlying capillary structures 19 with advantageously different pore sizes. The wick structure 19 arranged directly on the inlet side 61 of the heating body 60 prevents the formation of bubbles on the inlet side 61 of the heating body 60, since gas bubbles prevent a further feeding effect and at the same time cause (local) overheating of the heating body 60 due to a lack of cooling by liquid flowing in.

The liquid reservoir 18 can advantageously be at least partially filled by a liquid buffer element 51. The liquid buffer element 51 is advantageously arranged in contact with the wick structure 19. The liquid buffer element 51 is arranged to store liquid 50 from the liquid reservoir 18 and to transport it to the wick structure 19. This enables the wick structure 19 to be reliably supplied with liquid 50 from the liquid reservoir 18 irrespective of position or orientation. Like the wick structure 19, the liquid buffer element 51 can consist of one of the porous and/or capillary liquid-conducting materials described.

FIG. 7 shows a schematic cross section of a consumption unit 17 according to one embodiment of the invention.

The liquid reservoir 18 comprises a circular cross-section. Along the longitudinal axis, perpendicular to the figure plane, the jacket surface 104 extends with the wall 101 and the vent 5 with the flow channel 8. The vent 5 advantageously comprises a circular cross-section. The diameter of the vent 5 is smaller than the diameter of the liquid reservoir 18, which is advantageously defined by the diameter of the base surface 105. Advantageously, the vent 5 and the liquid reservoir 18 are arranged concentrically. However, in other embodiments, the vent 5 or the flow channel 8 may also be centered and/or arranged outside the area center of the cross-section of the fluid reservoir 18.

The vaporizer device 1 arranged in the liquid reservoir 18, respectively the carrier 4 of the vaporizer device 1 comprise an oval outer cross-section. In this example, the outer cross-section is elliptical. The elliptical outer cross-section of the vaporizer device 1 or of the carrier 4 comprises a large semi-axis, which is equal to the radius of the liquid reservoir 18. As a result, the vaporizer device 1 or the carrier 4 is supported in the liquid reservoir 18 at two contacting points 120, 121 diametrically opposite on the major axis of the ellipse. Between the wall 101 and the vaporizer device 1 or the carrier 4 there are two openings diametrically opposite on the minor axis of the ellipse, for example the filling opening 6 and a further opening 83 serving for venting. The opposite openings 6, 83 are closed in a liquid-tight manner by the first closure part 7 (not shown in FIG. 7).

In order to support an advantageous assembly of the consumption unit 17, in particular an advantageous alignment of the liquid reservoir 18, the vaporizer device 1 resp. the carrier 4 and/or the first closure part 7 during assembly, at least one guide element not shown, such as a nose, recess, groove, chamfer and/or a similar element suitable for guiding, can be provided, which prevents, for example, an unintentional rotation of the components.

The vaporizer device 1 preferably comprises an elongated shape. Insofar as the inhaler 10 and/or the liquid reservoir 18 also comprise an elongated shape, the vaporizer device is preferably aligned with its longitudinal axis parallel to the longitudinal axis of the inhaler 10 resp. the liquid reservoir 18 and thus also to the flow direction of the liquid to be vaporized, so that the liquid flows along the vaporizer device 1 over a flow path that is as long as possible. The liquid reservoir 18 can then be filled either parallel to the longitudinal axis of the vaporizer device 1 or perpendicular to the longitudinal axis of the vaporizer device 1, depending on the position of the filling opening 6.

1. A consumption unit for an electronic inhaler, comprising:
a liquid reservoir,
a vaporizer device arranged in the liquid reservoir, and
a vent with a flow channel extending in the liquid reservoir up to a flow connection of the flow channel to the environment,
wherein the liquid reservoir comprises a filling opening which is closed via a first closure part.

2. The consumption unit according to claim 1,
wherein the filling opening is formed by a free space between the vaporizer device and a wall of the liquid reservoir.

3. The consumption unit according to claim 1,
wherein the liquid reservoir comprises a cross-section perpendicular to a longitudinal direction of the flow channel which is larger than an outer cross-section formed by the vaporizer device perpendicular to the longitudinal direction of the flow channel, and
wherein the filling opening is provided between the inner contour of the liquid reservoir and the outer contour of the vaporizer device.

4. The consumption unit according to claim 3,
wherein the inner cross-sectional area of the liquid reservoir is circular and the outer cross-sectional area of the vaporizer device is oval.

5. The consumption unit according to claim 1,
wherein the filling opening is formed by a bore in a wall of the liquid reservoir.

6. The consumption unit according to claim 1,
wherein a venting opening is provided, which is closed via a second closure part.

7. The consumption unit according to claim 1,
wherein the filling opening is provided between the vent and a wall of the liquid reservoir.

8. The consumption unit according to claim 1,
wherein the vent and the vaporizer device and/or the vent and the liquid reservoir are formed at least in sections as a one-piece component.

9. The consumption unit according to claim 8,
wherein the liquid reservoir and/or the vaporizer device are molded with a portion which at least partially forms the vent.

10. The consumption unit according to claim 1,
wherein the first closure part fixes the vaporizer device relative to the liquid reservoir.

11. The consumption unit according to claim 10,
wherein the first closure part comprises a coupling geometry adapted to the geometry of the liquid reservoir and/or the vaporizer device.

12. The consumption unit according to claim 1,
wherein the vent is fluidically connected to an outlet side of the vaporizer device.

13. The consumption unit according to claim 1,
wherein the first closure part is designed as a mouthpiece.

14. The consumption unit according to claim 13,
wherein the first closure part comprises a flow channel which connects the flow channel of the vent to the environment.

15. The consumption unit according to claim 1,
wherein a wick structure is provided between the liquid reservoir and the vaporizer device, and
wherein the wick structure is oriented and/or connected to the liquid reservoir such that the feed of liquid through the wick structure is perpendicular or at an angle not equal to 180 degrees to the longitudinal extension of the vaporizer device.

16. An electronic inhaler comprising a consumption unit according to claim 1.

17. A method for manufacturing a consumption unit for an inhaler having a liquid reservoir, a vaporizer device, and a first closure part,
comprising the following process steps in the following order:
inserting the vaporizer device into the liquid reservoir to a final fastening position,
filling the liquid reservoir with liquid through a filling opening, and
closing the filling opening via the first closure part.

18. The method according to claim 17,
wherein the vaporizer device comprises a smaller outer geometry perpendicular to an insertion direction than a free cross-sectional area of the liquid reservoir perpendicular to the insertion direction of the vaporizer device, and
wherein the filling opening is formed by a free space which is formed due to the smaller outer geometry of the vaporizer device between the vaporizer device and the liquid reservoir.
